(19) United States
(12) Patent Application Publication
(10) Pub. No.: US 2024/0315941 A1
(43) Pub. Date: Sep. 26, 2024

(54) Oral Care Compositions and Methods of Use

(71) Applicant: Colgate-Palmolive Company, New York, NY (US)

(72) Inventors: Xiao Yi Huang, Guangzhou (CN); Michael Prencipe, West Windsor, NJ (US); Amy Russo, Belle Mead, NJ (US); Hansruedi Stettler, Basel (CH); Vyoma Patel, Hillsborough, NJ (US); Betty Won, Princeton Junction, NJ (US); Peng Yan, Guangzhou (CN); André Morgan, Robbinsville, NJ (US)

(73) Assignee: Colgate-Palmolive Company, New York, NY (US)

(21) Appl. No.: 18/594,760

(22) Filed: Mar. 4, 2024

Related U.S. Application Data
(63) Continuation of application No. 15/548,468, filed on Aug. 3, 2017, now abandoned.
(60) Provisional application No. 62/187,801, filed on Jul. 1, 2015.

(51) Int. Cl. A61K 8/44; A61K 8/21; A61K 8/25; A61K 8/27; A61K 8/34; A61K 8/362; A61K 8/365; A61Q 11/00

(52) U.S. Cl. CPC A61K 8/44; A61K 8/21; A61K 8/25; A61K 8/27; A61K 8/345; A61K 8/362; A61K 8/365; A61Q 11/00; A61K 2800/28; A61K 2800/92

(57) ABSTRACT

This invention relates to oral care compositions comprising arginine or lysine, zinc citrate and zinc oxide, a fluoride source, and glycerin, as well as to methods of using and of making these compositions.

### CROSS REFERENCE TO RELATED APPLICATIONS

This application is a continuation of U.S. Nonprovisional patent application Ser. No. 15/548,468, filed on Aug. 3, 2017, which is a U.S. National Stage Application of PCT Patent Application No. PCT/CN2016/086995, filed on Jun. 24, 2016, which claims the benefit of U.S. Provisional Patent Application No. 62/187,801, filed on Jul. 1, 2015, the contents of each are incorporated herein by reference in their entireties.

### FIELD

This invention relates to oral care compositions comprising arginine or lysine or salt thereof, zinc oxide and zinc citrate, and a fluoride source, as well as to methods of using and of making these compositions.

### BACKGROUND

Oral care compositions present particular challenges in preventing microbial contamination. Arginine and other basic amino acids have been proposed for use in oral care and are believed to have significant benefits in combating cavity formation and tooth sensitivity.

Commercially available arginine-based toothpaste contains arginine bicarbonate and precipitated calcium carbonate. The carbonate ion is believed to have cariostatic properties, and the calcium is believed to form in complex with arginine to provide a protective effect.

The formulation of certain oral care compositions presents special challenges. For example, not all preservatives are active at higher pH. Some preservatives negatively affect the taste or aesthetics of the product. While certain preservatives, such as ethanol or parabens, are known to be effective at a range of pHs, these preservatives are not suitable for all products or all markets.

Zinc formulations also present challenges. Zinc is a known antimicrobial agent used in toothpaste compositions. Zinc is also a known essential mineral for human health, and has been reported to help strengthen dental enamel and to promote cell repair. Unfortunately, conventional toothpaste formulations often require a high concentrations of zinc, e.g., 2% by weight or more, to achieve efficacy. At this concentration, the zinc imparts a notably astringent taste to the composition.

Foaming perception is also an important aspect of a user's experience and can be critical to the aesthetic and qualitative qualities of the oral care composition.

Accordingly, there is a need for improved foaming agents for use in oral compositions comprising basic amino acids and zinc salts.

### BRIEF SUMMARY

It has been surprisingly found that particular concentrations and ratios of glycerin—used as a humectant—can play a positive role in foam perception in oral care compositions with a zinc citrate, zinc oxide, and amino acid base. Without being bound by theory, glycerin can form weak hydrogen-bonds with water on the surface of the surfactant micelle, reducing water evaporation and minimizing bubble coalescence. In some aspects, the glycerin can be used as the sole humectant or in a particular ratio with sorbitol. This unique humectant blend (whether it is glycerin alone in a particular amount, a ratio of glycerin and sorbitol) has an unexpected positive impact on foam generation and foam density perception in oral care compositions.

In one aspect the invention is an oral care composition (Composition 1.0) comprising:
a. Basic amino acid in free or salt wherein the amino acid is selected from arginine, lysine, and combinations thereof (e.g., free form arginine)
b. zinc oxide and zinc citrate
c. a fluoride source (e.g., sodium fluoride); and
d. glycerin (e.g., from 26-35 wt. %)
   For example, the invention contemplates any of the following compositions (unless otherwise indicated, values are given as percentage of the overall weight of the composition)
1.1 Composition 1.0 wherein the basic amino acid has the L-configuration (e.g., L-arginine).
1.2 Any of the preceding compositions wherein the basic amino acid is arginine in free form.
1.3 Any of the preceding compositions wherein the basic amino acid is provided in the form of a di- or tri-peptide comprising arginine, or salts thereof.
1.4 Any of the preceding compositions wherein the basic amino acid is arginine, and wherein the arginine is present in an amount corresponding to 1% to 15%, e.g., 3 wt. % to 10 wt. % of the total composition weight, about e.g., 1.5%, 4%, 5%, or 8%, wherein the weight of the basic amino acid is calculated as free form.
1.5 Any of the preceding compositions wherein the amino acid is arginine from 0.1 wt. %-6.0 wt. % (e.g., about 1.5 wt. %).
1.6 Any of the preceding compositions wherein the amino acid is arginine from about 1.5 wt. %.
1.7 Any of the preceding compositions wherein the amino acid is arginine from 4.5 wt. %-8.5 wt. % (e.g., 5.0%).
1.8 Any of the preceding compositions wherein the amino acid is arginine from about 5.0 wt. %.
1.9 Any of the preceding compositions wherein the amino acid is arginine from 3.5 wt. %-9 wt. %.
1.10 Any of the preceding compositions wherein the amino acid is arginine from about 8.0 wt. %.
1.11 Any of the preceding compositions wherein the amino acid is L-arginine.
1.12 Any of the preceding compositions wherein the amino acid is free form arginine.
1.13 Any of the preceding compositions wherein the amino acid is arginine.
1.14 Composition 1.19 wherein the amino acid is arginine phosphate.
1.15 Composition 1.19 wherein the amino acid is arginine hydrochloride.
1.16 Composition 1.19 wherein the amino acid is arginine bicarbonate.
1.17 Any of the preceding compositions wherein the amino acid is arginine ionized by neutralization with an acid or a salt of an acid.
1.18 Any of preceding compositions wherein the composition is ethanol-free.
1.19 Any of the preceding compositions further comprising a fluoride source selected from: stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof.
1.20 Any of the preceding compositions wherein the fluoride source is sodium fluoride.
1.21 Any of the preceding compositions wherein the fluoride source is a fluoride salt present in an amount of 0.1 wt. % to 2 wt. % (0.1 wt. %-0.6 wt. %) of the total composition weight (e.g., sodium fluoride (e.g., about 0.32 wt. % 0).
1.22 Any of the preceding compositions wherein the fluoride source is a soluble fluoride salt which provides fluoride ion in an amount of from 50 to 25,000 ppm (e.g., 750-2000 ppm, e.g., 1000-1500 ppm, e.g., about 1000 ppm, e.g., about 1450 ppm).
1.23 Any of the preceding compositions wherein the fluoride source is sodium fluoride which provides fluoride in an amount from 750-2000 ppm (e.g., about 1450 ppm).
1.24 Any of the preceding compositions wherein the fluoride source is sodium fluoride and which provides fluoride in an amount from 1000 ppm-1500 ppm.
1.25 Any of the preceding compositions wherein the fluoride source is sodium fluoride and which provides fluoride in an amount of about 1450 ppm.
1.26 Any of the preceding compositions wherein the pH is between 7.5 and 10.5, e.g., 8-9.5, e.g., 7.2-9.0, about 8.0, about 9.0.
1.27 Any of the preceding compositions further comprising calcium carbonate.
1.28 The composition of 1.38, wherein the calcium carbonate is a precipitated calcium carbonate high absorption (e.g., 20% to 30% by weight of the composition) (e.g., 25% precipitated calcium carbonate high absorption).
1.29 The composition of 1.35, further comprising a precipitated calcium carbonate-light (e.g., about 10% precipitated calcium carbonate-light) (e.g., about 10% natural calcium carbonate).
1.30 Any of the preceding compositions further comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophosphate, monosodium phosphate, pentapotassium triphosphate and mixtures of any of two or more of these, e.g., in an amount of 0.1-20%, e.g., 0.1-8%, e.g., e.g., 0.2 to 5%, e.g., 0.3 to 2%, e.g., 0.3 to 1%, e.g about 0.5%, about 1%, about 2%, about 5%, about 6%, by weight of the composition.
1.31 Any of the preceding compositions comprising tetrapotassium pyrophosphate, disodium hydrogenorthophosphate, monosodium phosphate, and pentapotassium triphosphate.
1.32 Any of the preceding compositions comprising a polyphosphate.
1.33 The composition of 1.43, wherein the polyphosphate is tetrasodium pyrophosphate.
1.34 The composition of 1.44, wherein the tetrasodium pyrophosphate is from 0.1-1.0 wt. % (e.g., about 0.5 wt. %).
1.35 Any of the preceding compositions further comprising an abrasive or particulate (e.g., silica).
1.36 Any of the preceding compositions wherein the silica is synthetic amorphous silica. (e.g., 1%-28% by wt.) (e.g., 8%-25% by wt.).
1.37 Any of the preceding composition wherein the silica abrasives are silica gels or precipitated amorphous silicas, e.g., silicas having an average particle size ranging from 2.5 microns to 12 microns.
1.38 Any of the preceding compositions further comprising a small particle silica having a median particle size (d50) of 1-5 microns (e.g., 3-4 microns) (e.g., about 5 wt. % Sorbosil AC43 from PQ Corporation, Warrington, United Kingdom).
1.39 Any of the preceding compositions wherein 20-30 wt. % of the total silica in the composition is small particle silica (e.g., having a median particle size (d50) of 3-4 microns) and wherein the small particle silica is about 5 wt. % of the oral care composition.
1.40 Any of the preceding compositions comprising silica wherein the silica is used as a thickening agent, e.g., particle silica.
1.41 Any of the preceding compositions further comprising a nonionic surfactant, wherein the nonionic surfactant is in an amount of from 0.5-5%, e.g, 1-2%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof.
1.42 Any of the preceding compositions, wherein the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407.
1.43 Any of the preceding compositions, wherein the ratio of the amount of zinc oxide (e.g., wt. %) to zinc citrate (e.g., wt. %) is from 1.5:1 to 4.5:1 (e.g., 2:1, 2.5:1, 3:1, 3.5:1, or 4:1).
1.44 Any of the preceding compositions, wherein the zinc citrate is in an amount of from 0.25 to 1.0 wt. % (e.g., 0.5 wt. %) and zinc oxide may be present in an amount of from 0.75 to 1.25 wt. % (e.g., 1.0 wt. %) based on the weight of the oral care composition.
1.45 Any of the preceding compositions wherein the zinc citrate is about 0.5 wt. %.
1.46 Any of the preceding compositions wherein the zinc oxide is about 1.0 wt. %.
1.47 Any of the preceding compositions where the zinc citrate is about 0.5 wt. % and the zinc oxide is about 1.0 wt. %.
1.48 Any of the preceding compositions further comprising an additional ingredient selected from: benzyl alcohol, Methylisothizolinone (“MIT”), Sodium bicarbonate, sodium methyl cocoyl taurate (tauranol), lauryl alcohol, and polyphosphate.
1.49 Any of the preceding compositions wherein the benzyl alcohol is present from 0.1-0.6 wt. %., (e.g., 0.1-0.4 wt. %) e.g., about 0.1 wt. %, about 0.2 wt. %, or about 0.3 wt. %.
1.50 Any of the preceding compositions wherein the benzyl alcohol is about 0.1 wt. %.
1.51 Any of the preceding composition wherein benzyl alcohol is present at is considered a preservative.
1.52 Any of the preceding compositions comprising polymer films.
1.53 Any of the preceding compositions comprising flavoring, fragrance and/or coloring.
1.54 The composition of 1.42, wherein the flavoring agent is sodium saccharin, sucralose, or a mixture thereof.
1.55 Any of the preceding compositions, wherein the composition comprises a thickening agents selected from the group consisting of carboxyvinyl polymers, carrageenan, xanthan, hydroxyethyl cellulose and water-soluble salts of cellulose ethers (e.g., sodium carboxymethyl cellulose and sodium carboxymethyl hydroxyethyl cellulose).
1.56 Any of the preceding compositions, wherein the compositions comprise sodium carboxymethyl cellulose (e.g., from 0.5 wt. %-1.5 wt. %)
1.57 Any of the preceding compositions comprising from 5%-40%, e.g., 10%-35%, e.g., about 15%, 25%, 30%, and 35% water.
1.58 Any of the preceding compositions comprising an additional antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, Zinc Chloride, Zinc Lactate, Zinc Sulfate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.
1.59 Any of the preceding compositions comprising an antioxidant, e.g., selected from the group consisting of Co-enzyme Q10, PQQ, Vitamin C, Vitamin E, Vitamin A, BHT, anethole-dithiothione, and mixtures thereof.
1.60 Any of the preceding compositions comprising a whitening agent.
1.61 Any of the preceding compositions comprising a whitening agent selected from a whitening active selected from the group consisting of peroxides, metal chlorites, perborates, percarbonates, peroxyacids, hypochlorites, and combinations thereof.
1.62 Any of the preceding compositions further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or hydrogen peroxide polymer complexes such as hydrogen peroxide-polyvinyl pyrrolidone polymer complexes.
1.63 Any of the preceding compositions, wherein the glycerin is in an amount from 20%-40% by wt. of the composition.
1.64 The composition of 1.62, wherein the glycerin is in an amount from 25%-40% by wt. of the composition.
1.65 The composition of 1.63, wherein the glycerin is about 26% by wt. of the composition.
1.66 The composition of 1.63, wherein the glycerin is about 35% by wt. of the composition.
1.67 Any of the preceding compositions, wherein the composition further comprises sorbitol.
1.68 The composition of 1.66, wherein the sorbitol is from 10%-20% by wt. of the composition.
1.69 The composition of 1.67, wherein the sorbitol is about 13% by wt. of the composition.
1.70 The composition of 1.68, wherein the composition comprises about 13% by wt. sorbitol and about 26% by wt. glycerin.
1.71 Any of the preceding compositions further comprising an agent that interferes with or prevents bacterial attachment, e.g., ethyl lauroyl arginiate (ELA) or chitosan.
1.72 Any of the preceding compositions comprising:
   a. about 1.0% zinc oxide
   b. about 0.5% zinc citrate
   c. about 1.5% L-arginine
   d. about 0.32% sodium fluoride; and
   e. about 35% glycerin.
1.73 Any of compositions 1.0-1.81 comprising:
   a. about 1.0% zinc oxide
   b. about 0.5% zinc citrate
   c. about 5% L-arginine
   d. about 0.32% sodium fluoride;
   e. about 26% glycerin; and
   f. about 13% sorbitol.
1.74 Any of the preceding compositions effective upon application to the oral cavity, e.g., by rinsing, optionally in conjunction with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth, (xiii) clean the teeth and oral cavity (xiv) reduce erosion, (xv) prevents stains and/or whiten teeth, (xvi) immunize the teeth against cariogenic bacteria; and/or (xvii) promote systemic health, including cardiovascular health, e.g., by reducing potential for systemic infection via the oral tissues.
1.75 Any of the preceding oral compositions, wherein the oral composition may be any of the following oral compositions selected from the group consisting of: a toothpaste or a dentifrice, a mouthwash or a mouth rinse, a topical oral gel, and a denture cleanser.
1.76 A composition obtained or obtainable by combining the ingredients as set forth in any of the preceding compositions.

A composition obtained or obtainable by combining the ingredients as set forth in any of the preceding compositions.

A composition for use as set for in any of the preceding compositions.

In another embodiment, the invention encompasses a method to improve oral health comprising applying an effective amount of the oral composition of any of the embodiments set forth above to the oral cavity of a subject in need thereof, e.g., a method to
i. reduce or inhibit formation of dental caries,
ii. reduce, repair or inhibit early enamel lesions, e.g., as detected by quantitative light-induced fluorescence (QLF) or electrical caries measurement (ECM),
iii. reduce or inhibit demineralization and promote remineralization of the teeth,
iv. reduce hypersensitivity of the teeth,
v. reduce or inhibit gingivitis,
vi. promote healing of sores or cuts in the mouth,
vii. reduce levels of acid producing bacteria,
viii. to increase relative levels of arginolytic bacteria,
ix. inhibit microbial bio film formation in the oral cavity,
x. raise and/or maintain plaque pH at levels of at least pH 5.5 following sugar challenge,
xi. reduce plaque accumulation,
xii. treat dry mouth,
xiii. enhance systemic health, including cardiovascular health, e.g., by reducing potential for systemic infection via the oral tissues,
xiv. Whiten teeth,
xv. reduce erosion of the teeth,
xvi. immunize (or protect) the teeth against cariogenic bacteria and their effects, and/or
xvii. clean the teeth and oral cavity.
The invention further comprises the use of sodium bicarbonate, sodium methyl cocoyl taurate (tauranol), MIT, and benzyl alcohol and combinations thereof in the manufacture of a Composition of the Invention, e.g., for use in any of the indications set forth in the above method of Composition 1.0, et seq.

### DETAILED DESCRIPTION

As used herein, the term “oral composition” means the total composition that is delivered to the oral surfaces. The composition is further defined as a product which, during the normal course of usage, is not, the purposes of systemic administration of particular therapeutic agents, intentionally swallowed but is rather retained in the oral cavity for a time sufficient to contact substantially all of the dental surfaces and/or oral tissues for the purposes of oral activity. Examples of such compositions include, but are not limited to, toothpaste or a dentifrice, a mouthwash or a mouth rinse, a topical oral gel, a denture cleanser, and the like.

As used herein, the term “dentifrice” means paste, gel, or liquid formulations unless otherwise specified. The dentifrice composition can be in any desired form such as deep striped, surface striped, multi-layered, having the gel surrounding the paste, or any combination thereof. Alternatively the oral composition may be dual phase dispensed from a separated compartment dispenser.

### Basic Amino Acids

The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

Accordingly, basic amino acids include, but are not limited to, arginine, lysine, serine, citrullene, ornithine, creatine, histidine, diaminobutanoic acid, diaminoproprionic acid, salts thereof or combinations thereof. In a particular embodiment, the basic amino acids are selected from arginine, citrullene, and ornithine.

In certain embodiments, the basic amino acid is arginine, for example, L-arginine, or a salt thereof.

The compositions of the invention (e.g., Composition 1.0 et seq) are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

### Fluoride Ion Source

The oral care compositions (e.g., Composition 1.0 et seq) may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference. Representative fluoride ion sources used with the present invention (e.g., Composition 1.0 et seq.) include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

### Surfactants

The invention may in some embodiments contain anionic surfactants, e.g., the Compositions of Composition 1.0, et seq., for example, water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOS0₃X, wherein m is 6-16, e.g., 10, n is 1-6, e.g., 2, 3 or 4, and X is Na or, for example sodium laureth-2 sulfate (CH₃(CH2)₁₀CH₂(OCH₂CH₂)₂OS0₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆-₃₀ alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., 1.5%.

In another embodiment, cationic surfactants useful in the present invention can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants of Composition 1.0, et seq., that can be used in the compositions of the invention can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the invention comprises a nonionic surfactant selected from poloxamers (e.g., poloxamer 407), polysorbates (e.g., polysorbate 20), polyoxyl hydrogenated castor oils (e.g., polyoxyl 40 hydrogenated castor oil), and mixtures thereof.

Illustrative amphoteric surfactants of Composition 1.0, et seq., that can be used in the compositions of the invention include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials.

The surfactant or mixtures of compatible surfactants can be present in the compositions of the present invention (e.g., Composition 1.0 et seq) in 0.1% to 5%, in another embodiment 0.3% to 3% and in another embodiment 0.5% to 2% by weight of the total composition.

### Flavoring Agents

The oral care compositions of the invention may also include a flavoring agent. Flavoring agents which are used in the practice of the present invention include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition (e.g., Composition 1.0 et seq) at a concentration of 0.01 to 2% by weight.

### Chelating and Anti-Calculus Agents

The oral care compositions of the invention also may include one or more chelating agents able to complex calcium found in the cell walls of the bacteria. Binding of this calcium weakens the bacterial cell wall and augments bacterial lysis.

Another group of agents suitable for use as chelating or anti-calculus agents in the present invention are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide least 0.1 wt. % pyrophosphate ions, e.g., 0.1 to 3 wt. % 5, e.g., 0.1 to 2 wt. %, e.g., 0.1 to 1 wt. %, e.g., 0.2 to 0.5 wt. %. The pyrophosphates also contribute to preservation of the compositions by lowering water activity.

### Polymers

The oral care compositions of the invention also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water-soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available, for example as Gantrez AN 139 (M.W. 500,000), AN 119 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalkane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

Another useful class of polymeric agents includes polyamino acids, particularly those containing proportions of anionic surface-active amino acids such as aspartic acid, glutamic acid and phosphoserine, as disclosed in U.S. Pat. No. 4,866,161 Sikes et al., incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, xanthan gum, hydroxyethyl cellulose and water-soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

### Abrasives

Natural calcium carbonate is found in rocks such as chalk, limestone, marble and travertine. It is also the principle component of egg shells and the shells of mollusks. The natural calcium carbonate abrasive of the invention is typically a finely ground limestone which may optionally be refined or partially refined to remove impurities. For use in the present invention, the material has an average particle size of less than 10 microns, e.g., 3-7 microns, e.g., about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8=4.9, e.g., about 4.3; a D50 of 1-4 microns, e.g., 2.2-2.6 microns, e.g., about 2.4 microns, and a D10 of 1-2 microns, e.g., 1.2-1.4, e.g., about 1.3 microns. The particles have relatively high water absorption, e.g., at least 25 g/100 g, e.g., 30-70 g/100 g. Examples of commercially available products suitable for use in the present invention include, for example, Carbolag® 15 Plus from Lagos Industria Quimica.

In certain embodiments the invention may comprise additional calcium-containing abrasives, for example calcium phosphate abrasive, e.g., tricalcium phosphate (Ca₃(P0₄)₂), hydroxyapatite (Ca₁₀(P0₄)₆(OH)₂), or dicalcium phosphate dihydrate (CaHP0₄·2H₂0, also sometimes referred to herein as DiCal) or calcium pyrophosphate, and/or silica abrasives, sodium metaphosphate, potassium metaphosphate, aluminum silicate, calcined alumina, bentonite or other siliceous materials, or combinations thereof. Any silica suitable for oral care compositions may be used, such as precipitated silicas or silica gels. For example, synthetic amorphous silica. Silica may also be available as a thickening agent, e.g., particle silica. For example, the silica can also be small particle silica (e.g., Sorbosil AC43 from PQ Corporation, Warrington, United Kingdom). However, the additional abrasives are preferably not present in a type or amount so as to increase the RDA of the dentifrice to levels which could damage sensitive teeth, e.g., greater than 130.

### Water

Water is present in the oral compositions of the invention. Water, employed in the preparation of commercial oral compositions should be deionized and free of organic impurities. Water commonly makes up the balance of the compositions and includes 5% to 45%, e.g., 10% to 20%, e.g., 25-35%, by weight of the oral compositions. This amount of water includes the free water which is added plus that amount which is introduced with other materials such as with sorbitol or silica or any components of the invention. The Karl Fischer method is a one measure of calculating free water.

### Humectants

Within certain embodiments of the oral compositions, it is also desirable to incorporate a further humectant (e.g., in addition to glycerin) to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerin, sorbitol, xylitol, propylene glycol as well as other polyols and mixtures of these humectants. Mixtures of glycerin and sorbitol may be used in certain embodiments as the humectant component of the compositions herein (e.g., Composition 1.0 et seq).

The present invention in its method aspect involves applying to the oral cavity a safe and effective amount of the compositions described herein.

The compositions and methods according to the invention (e.g., Composition 1.0 et seq) can be incorporated into oral compositions for the care of the mouth and teeth such as toothpastes, transparent pastes, gels, mouth rinses, sprays and chewing gum.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### Example 1

Combinations of humectants (single and mixed, with net humectant levels of about 35%) are evaluated for foam analysis (Foam Volume) using the SITA Foam Tester R-2000 at 40 Seconds.

Samples are prepared as toothpaste slurries. While mixing this slurry, minimum foam is generated. The slurry is poured into a jacketed glass beaker each time. To ensure that the slurry does not separate, the mixture is continuously stirred on a magnetic stir plate. Once the program starts, 250 mL of the slurry is transferred via a rubber hose. Once the desired amount is reached, needles measure the baseline and then, the rotor speeds up to 800 rpm. The volume of foam generated is measured every 10 s for 40 s by the needles. The foam volume at 40 s is determined as the average over all replicates.

Foam density is assessed visually while the SITA foam tester is measuring foam decay at 10 s intervals after the Foam Generation procedure, the Foam Tester will measure foam decay at 10 s second intervals 6 times. At a set time interval the following values are considered: foam volume given by the SITA instrument; actual liquid volume. With these observations and knowing the original test volume and the density of the test slurry, foam density is calculated. Results are demonstrated in Table 1.

TABLE 1
(Foam Volume)

| | Description | Net | Avg. Foam | |
|---|---|---|---|---|
| | (% listed by | Humectant | Volume | Std. |
| Formulation | wt. of composition) | (%) | (ml) | Dev. |
| Formula A | ZnO 1.0%, ZnCit 0.5%, | 34.5 | 219 | 3 |
| | Arginine 1.5%, | | | |
| | Sorbitol 45%, | | | |
| | Glycerin 3% | | | |
| Formula B | ZnO 1.0%, ZnCit 0.5%, | 34 | 198 | 10 |
| | Arginine 1.5%, | | | |
| | Sorbitol 20%, | | | |
| | Glycerin 20% | | | |
| Formula C | ZnO 1.0%, ZnCit 0.5%, | 35.1 | 306 | 8 |
| | Arginine 1.5% | | | |
| | Sorbitol 13%, | | | |
| | Glycerin 26% | | | |
| Formula D | ZnO 1.0%, ZnCit 0.5%, | 35 | 294 | 25 |
| | Arginine 1.5% | | | |
| | Glycerin 35% | | | |

Formulations A and B contain sorbitol (70% in solution) at amounts from 20-45% by wt. of the total composition.

Formulations C and D contain sorbitol (70% in solution) at amounts from 0-13% by wt. The glycerin ranges from 26%-35% in these formulations. Foam volume of Formulations C and D is significantly increased than either Formulation A or B.

### Example 2

Combinations of humectants (single and mixed, with net humectant levels of about 35%) are evaluated for foam analysis (Foam Density) using the SITA Foam Tester R-2000 at 40 Seconds. Results are demonstrated in Table 2.

TABLE 2

| | | Net | Avg. Foam | |
|---|---|---|---|---|
| | | Humectant | Density | Std. |
| Formulation | Description | (%) | (g/ml) | Dev. |
| Formula E | ZnO 1.0%, ZnCit 0.5%, | 34.5 | 0.275 | 0.005 |
| | Arginine 1.5% | | | |
| | Sorbitol 45%, | | | |
| | Glycerin 3% | | | |
| Formula F | ZnO 1.0%, ZnCit 0.5%, | 35 | 0.361 | 0.006 |
| | Arginine 1.5% | | | |
| | Glycerin 35% | | | |

Formula E comprises 45% by wt of the composition sorbitol (70% in solution) and 3% glycerin, and Formula F comprises 35% glycerin without the addition of any sorbitol. The foam density of Formula F (glycerin 35%) shows a clear increase over the foam density of Formula E wherein the sorbitol amount by wt. of the composition, is used in far greater amounts than the glycerin.

Examples 1 and 2 demonstrate increased foam volume and foam density in formulations which employ glycerin in an amount from 26%-35% by wt. (as compared to the representative sorbitol formulations).

### Example 3

In one representative formulation, a dentifrice comprises the following:
a. 1.0 wt. % zinc oxide
b. 0.5 wt. % zinc citrate
c. 1.5 wt. % L-arginine
d. 0.32 wt. % sodium fluoride; and
e. 35% wt. glycerin
   Wherein the dentifrice is expected to have increased foam volume and density compared to formulations which employ between 20%-45% sorbitol.

### Example 4

### Representative Dentifrice Formulations

The data from the following formulations is detailed in above Example 1 and Example 2.

| Ingredient | Formulation A (wt. %) |
|---|---|
| 99.0%-101.0% GLYCERIN-USP, EP VEG | 3 |
| POLYMERS | 1.1 |
| FLAVORING AGENTS | 2 |
| SODIUM FLUORIDE-USP, EP | 0.32 |
| ALKALI PHOSPHATE SALT | 0.5 |
| SORBITOL-NON-CRYSTAL-70% SOLN USP, | 45 |
| DEMINERALIZED WATER | Q.S. |
| ZINC CITRATE TRIHYDRATE | 0.5 |
| L-ARGININE | 1.5 |
| 85% SYRUPY PHOSPHORIC ACID-FOOD GRADE | 0.35 |
| ZINC OXIDE | 1 |
| COLORANT | 0.75 |
| ABRASIVES | 20 |
| SILICA-THICKENER | 4.5 |
| AMPHOTERIC SURFACTANT | 1.25 |
| NON-IONIC SURFACTANTS | 0.5 |
| ANIONIC SURFACTANT | 2 |
| PRESERVATIVE | 0.2 |
| Total Components | 100 |

| Ingredient | Formulation B (wt. %) |
|---|---|
| 99.0%-101.0% GLYCERIN-USP, EP VEG | 20 |
| POLYMERS | 1.1 |
| DEMINERALIZED WATER | Q.S. |
| FLAVORING AGENTS | 2 |
| SODIUM FLUORIDE-USP, EP | 0.32 |
| ALKALI PHOSPHATE SALT | 0.5 |
| SORBITOL | 20 |
| ZINC CITRATE TRIHYDRATE | 0.5 |
| L-ARGININE | 1.5 |
| 85% SYRUPY PHOSPHORIC ACID-FOOD GRADE | 0.35 |
| ZINC OXIDE | 1 |
| COLORANT | 0.75 |
| ABRASIVES | 20 |
| THICKENER SILICA | 4.5 |
| AMPHOTERIC SURFACTANT | 1.25 |
| NON-IONIC SURFACTANTS | 0.5 |
| ANIONIC SURFACTANT | 2 |
| PRESERVATIVE | 0.2 |
| Total Components | 100 |

| Ingredient | Formulation C (wt. %) |
|---|---|
| 99.0%-101.0% GLYCERIN-USP, EP VEG | 26 |
| POLYMERS | 1.1 |
| DEMINERALIZED WATER | Q.S. |
| FLAVORING AGENTS | 2 |
| SODIUM FLUORIDE-USP, EP | 0.32 |
| ALKALI PHOSPHATE SALT | 0.5 |
| SORBITOL NON-CRYSTAL-70% SOLN USP, EP | 13 |
| ZINC CITRATE TRIHYDRATE | 0.5 |
| L-ARGININE | 1.5 |
| 85% SYRUPY PHOSPHORIC ACID-FOOD GRADE | 0.35 |
| ZINC OXIDE | 1 |
| COLORANT | 0.75 |
| ABRASIVES | 20 |
| THICKENER SILICA | 4.5 |
| AMPHOTERIC SURFACTANT | 1.25 |
| NON-IONIC SURFACTANTS | 0.5 |
| ANIONIC SURFACTANT | 2 |
| PRESERVATIVE | 0.2 |
| Total Components | 100 |

| Ingredient | Formulation D (wt. %) |
|---|---|
| 99.0%-101.0% GLYCERIN-USP, EP VEG | 35 |
| POLYMERS | 1.1 |
| DEMINERALIZED WATER | Q.S. |
| FLAVORING AGENT | 2 |
| SODIUM FLUORIDE-USP, EP | 0.32 |
| ALKALI PHOSPHATE SALT | 0.5 |
| ZINC CITRATE TRIHYDRATE | 0.5 |
| L-ARGININE | 1.5 |
| 85% SYRUPY PHOSPHORIC ACID-FOOD GRADE | 0.35 |
| ZINC OXIDE | 1 |
| COLORANT | 0.75 |
| ABRASIVES | 20 |
| SILICA-THICKENER | 4.5 |
| AMPHOTERIC SURFACTANT | 1.25 |
| NON-IONIC SURFACTANTS | 0.5 |
| ANIONIC SURFACTANT | 2 |
| PRESERVATIVE | 0.2 |
| Total Components | 100 |

| Ingredient | Formulation E (wt. %) |
|---|---|
| 99.0%-101.0% GLYCERIN-USP, EP VEG | 3 |
| POLYMERS | 0.7 |
| FLAVORING AGENTS | 1.9 |
| SODIUM FLUORIDE-USP, EP | 0.32 |
| ALKALI PHOSPHATE SALT | 0.5 |
| SORBITOL | 45 |
| ABRASIVES | 20 |
| SILICA-THICKENER | 6.5 |
| ANIONIC SURFACTANT | 5.7 |
| AMPHOTERIC SURFACTANT | 1.25 |
| NON-IONIC SURFACTANTS | 0.5 |
| ZINC OXIDE | 1 |
| ZINC CITRATE TRIHYDRATE | 0.5 |
| COLORANT | 0.75 |
| PRESERVATIVE | 0.3 |
| 85% SYRUPY PHOSPHORIC ACID-FOOD GRADE | 0.35 |
| L-ARGININE | 1.5 |
| DEMINERALIZED WATER | Q.S. |
| Total Components | 100 |

| Ingredient | Formulation F (wt. %) |
|---|---|
| 99.0%-101.0% GLYCERIN-USP, EP VEG | 35 |
| POLYMERS | 1.2 |
| SODIUM FLUORIDE-USP, EP | 0.32 |
| ALKALI PHOSPHATE SALT | 0.5 |
| DEMINERALIZED WATER | Q.S. |
| ABRASIVES | 15 |
| SILICA-THICKENER | 6 |
| FLAVORING INGREDIENT | 1.9 |
| ANIONIC SURFACTANT | 5.7 |
| AMPHOTERIC SURFACTANT | 1.25 |
| NON-IONIC SURFACTANTS | 0.5 |
| ZINC CITRATE TRIHYDRATE | 0.5 |
| COLORANT | 0.75 |
| PRESERVATIVE | 0.4 |
| 85% SYRUPY PHOSPHORIC ACID-FOOD GRADE | 0.35 |
| L-ARGININE | 1.5 |
| ZINC OXIDE | 1 |
| Total Components | 100 |

### Example 5

### Representative Dentifrice Formulations

| Description | Formula 1 | Formula 2 | Formula 3 |
|---|---|---|---|
| DEMINERALIZED WATER | Q.S. | Q.S. | Q.S. |
| ABRASIVES | 10%-20% | 15%-20% | 15%-20% |
| 99.0%-101.0% GLYCERIN- | 35 | 35 | 35 |
| USP, EP VEG | | | |
| L-Arginine | 1.5 | 1.5 | 1.5 |
| AMPHOTERIC SURFACTANT | 1.0%-1.5% | 1.0%-1.5% | 1.0%-1.5% |
| NON-IONIC SURFACTANTS | 0.25%-0.75% | 0.25%-0.75% | 0.25%-0.75% |
| POLYMERS | 1.0%-1.4% | 0.75%-1.25% | 1.1%-1.5% |
| ALKALI PHOSPHATE SALT | 0.25%-0.75% | 0.25%-0.75% | 0.25%-0.75% |
| ZINC CITRATE TRIHYDRATE | 0.5 | 0.5 | 0.5 |
| COLORANT | 0.25%-1.0% | 0.25%-1.0% | 0.25%-1.0% |
| FLAVORING AGENTS | 1.5%-1.9% | 1.5%-1.9% | 1.5%-1.9% |
| 85% SYRUPY PHOSPHORIC | 0.35 | 0.35 | 0 |
| ACID-FOOD GRADE | | | |
| SODIUM FLUORIDE- | 0.32 | 0.32 | 0.32 |
| USP, EP | | | |
| SILICA-THICKENER | 5%-7% | 5%-7% | 6%-8% |
| ANIONIC SURFACTANT | 1%-3% | 1%-3% | 1%-3% |
| ZINC OXIDE | 1 | 1 | 1 |
| PRESERVATIVE | 0.4 | 0.4 | 0.4 |
| Total Components | 100 | 100 | 100 |

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

While the present invention has been described with reference to embodiments, it will be understood by those skilled in the art that various modifications and variations may be made therein without departing from the scope of the present invention as defined by the appended claims.

1. An oral care composition comprising:
a. a basic amino acid in free or salt form, wherein the basic amino acid is arginine, and wherein the arginine is present in an amount of 1.5 wt. % based on the weight of the total composition, the weight of the basic amino acid being calculated on the basis of the free form;
b. zinc oxide and zinc citrate, wherein the zinc citrate is in an amount of 0.5 wt. % and zinc oxide is in an amount of 1.0 wt. % based on the weight of the oral care composition;
c. a fluoride source;
d. glycerin, wherein the glycerin is in an amount of 35 wt. % based on the weight of the oral care composition, and wherein glycerin is the sole humectant.

2. The oral care composition of claim 1, wherein the basic amino acid is arginine and wherein the arginine has the L-configuration.

3. The oral care composition of claim 1 wherein the amino acid is arginine in free form.

4. The oral care composition of claim 1 wherein the amino acid is arginine in partially or wholly salt form.

5. The oral care composition of claim 1, wherein the fluoride source is sodium fluoride.

6. The oral care composition of claim 1 comprising:
a. 1.0% zinc oxide
b. 0.5% zinc citrate
c. 1.5% L-arginine
d. 0.32% sodium fluoride; and
e. 35% glycerin.

7. The oral care composition of claim 1, wherein the oral composition may be any of the following oral compositions selected from the group consisting of: a toothpaste or a dentifrice, a mouthwash or a mouth rinse, a topical oral gel, and a denture cleanser.

8. A method to improve oral health comprising applying an effective amount of the oral composition of claim 1 set forth above to the oral cavity of a subject in need thereof, wherein the method is effective to:
i. reduce or inhibit formation of dental caries,
ii. reduce, repair or inhibit early enamel lesions, e.g., as detected by quantitative light-induced fluorescence (QLF) or electrical caries measurement (ECM),
iii. reduce or inhibit demineralization and promote remineralization of the teeth,
iv. reduce hypersensitivity of the teeth,
v. reduce or inhibit gingivitis,
vi. promote healing of sores or cuts in the mouth,
vii. reduce levels of acid producing bacteria,
viii. to increase relative levels of arginolytic bacteria,
ix. inhibit microbial bio film formation in the oral cavity,
x. raise and/or maintain plaque pH at levels of at least pH 5.5 following sugar challenge,
xi. reduce plaque accumulation,
xii. treat dry mouth,
xiii enhance systemic health, including cardiovascular health,
xiv. Whiten teeth,
xv. reduce erosion of the teeth,
xvi. immunize (or protect) the teeth against cariogenic bacteria and their effects, and/or
xvii. clean the teeth and oral cavity.
